(19) United States
(12) Patent Application Publication
(10) Pub. No.: US 2016/0271412 A1
(43) Pub. Date: Sep. 22, 2016

(54) Cold Plasma Treatment System

(71) Applicant: Plasmology4, Inc., Scottsdale, AZ (US)

(72) Inventors: Robert M. Hummel, Cave Creek, AZ (US); Marc C. Jacofsky, Phoenix, AZ (US); Steven A. Myers, Scottsdale, AZ (US)

(21) Appl. No.: 15/072,284

(22) Filed: Mar. 16, 2016

Related U.S. Application Data
(60) Provisional application No. 62/134,401, filed on Mar. 17, 2015.

(51) Int. Cl. A61N 1/44; A61H 15/00

(52) U.S. Cl. CPC A61N 1/44; A61H 15/00; A61H 2015/0014; A61H 2201/0214

(57) ABSTRACT

A system including a cold plasma treatment system, including a controller configured to produce an electrical signal that generates cold plasma, a cold plasma applicator coupled to the controller, including a roller with at least one cold plasma generating region and at least one massage region, wherein the cold plasma applicator is configured to provide a cold plasma treatment and a massage treatment with the at least one cold plasma generating region and a massage treatment with the at least one massage region.

### CROSS-REFERENCE TO RELATED APPLICATIONS

This application claims priority to and benefit of U.S. Provisional Application No. 62/134,401 entitled “Cold Plasma Treatment System,” filed on Mar. 17, 2015, which is hereby incorporated by reference in its entirety.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Modern medicine enables physicians to treat a wide variety of injuries and infections. For example, physicians may treat these injuries and infections using topical medication (e.g., creams, foams, gels, ointments, bandages, etc.) and/or internal medication (e.g., medicine administered orally, intravenously). Unfortunately, existing treatments may be costly, ineffective, and/or slow to treat certain injuries and infections.

### BRIEF DESCRIPTION OF THE DRAWINGS

Various features, aspects, and advantages of the present invention will become better understood when the following detailed description is read with reference to the accompanying figures in which like characters represent like parts throughout the figures, wherein:

FIG. 1 is a side view of an embodiment of a cold plasma treatment system;

FIG. 2 is a perspective view of an embodiment of a cold plasma applicator;

FIG. 3 is a cross-sectional view of an embodiment of a roller assembly along line 3-3 of FIG. 2;

FIG. 4 is a side view of an embodiment of a cold plasma applicator;

FIG. 5 is a side view of an embodiment of a cold plasma applicator;

FIG. 6 is a side view of an embodiment of a cold plasma applicator; and

FIG. 7 is a side view of an embodiment of a cold plasma applicator.

### DETAILED DESCRIPTION OF SPECIFIC EMBODIMENTS

One or more specific embodiments of the present invention will be described below. These described embodiments are only exemplary of the present invention. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The disclosed embodiments include a cold plasma treatment system that enables simultaneous cold plasma and massage treatments. As will be explained in detail, the cold plasma treatment system may include a cold plasma applicator with one or more cold plasma generating regions and one or more massage treatment regions. In operation, the cold plasma applicator generates cold plasma at the cold plasma generation regions to treat a patient treatment site. The cold plasma may accelerate healing of the patient treatment site by killing bacteria, reducing inflammation, accelerating blood coagulation, facilitating the release of growth factors, etc. As the cold plasma generating regions produce cold plasma, the cold plasma applicator may be manipulated to move (e.g., slide, roll, etc.) over the patient treatment site. As the cold plasma applicator moves over the patient treatment site, the massage treatment regions provide a massaging treatment that increases blood flow, increases joint flexibility, relaxes injured and overused muscles, etc. Depending on the embodiment, the distribution of the cold plasma generating regions and massage treatment regions may vary along the length of the cold plasma applicator to provide different types of massage and cold plasma treatments. For example, the cold plasma applicator may include interchangeable rollers that have different cold plasma generating regions and massaging treatment regions that enable a customized treatment of a patient treatment site. In some embodiments, the cold plasma applicator may be flexible to facilitate conforming to a patient treatment site. The cold plasma applicator may also include vibration elements and/or heating elements to aid in treatment.

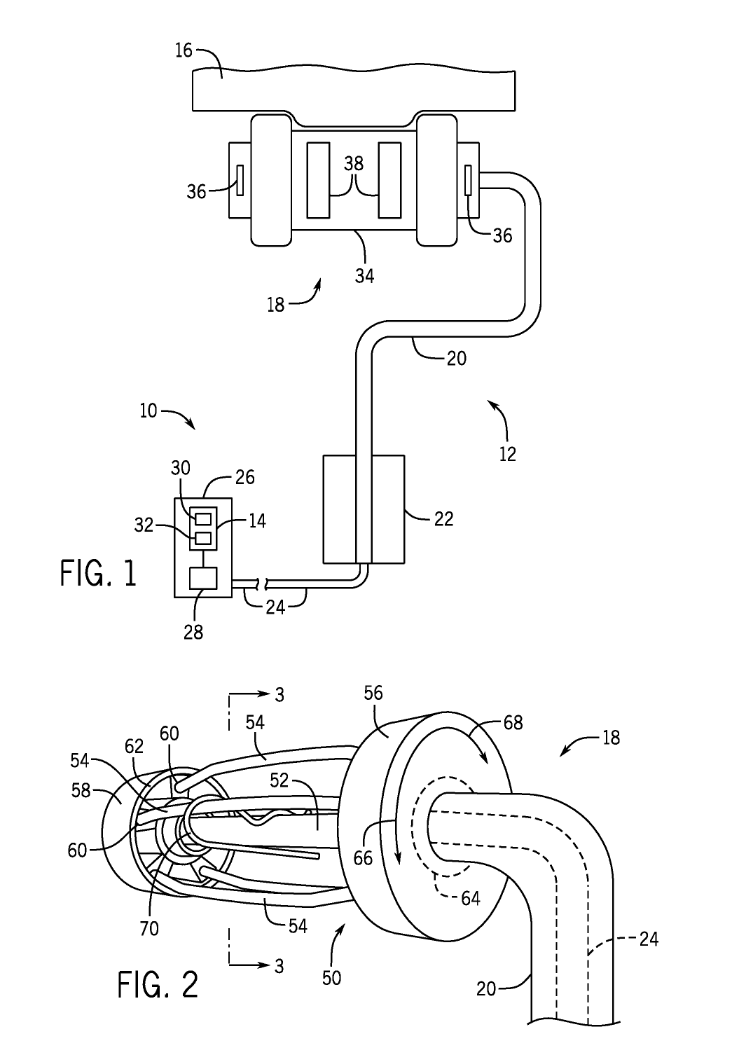

FIG. 1 is a side view of an embodiment of a cold plasma treatment system 10 (e.g., medical treatment system) with a cold plasma applicator 12 coupled to a controller 14. In operation, the cold plasma treatment system 10 is capable of generating cold plasma while simultaneously providing a massaging effect at a patient treatment site 16 (e.g., arm, leg, back, chest, etc.). As illustrated, the cold plasma applicator 12 may include a roller assembly 18 coupled to a handle 20. In some embodiments, the handle 20 may include a grip 22 to facilitate handling and increase comfort during use. In some embodiments, the handle 20 may be hollow enabling a data and/or electrical cable (e.g., wire 24) to pass through the handle 20 to the roller assembly 18. The wire 24 transfers an electrical signal from the controller 14 to the roller assembly 18 enabling the roller assembly 18 to produce a cold plasma during use. The cold plasma treatment system 10 may also be portable enabling use in an environment away from a medical facility (e.g., sports arena). Accordingly, the cold plasma treatment system 10 may include a portable housing or module 26 that contains the controller 14 and a power source 28 (e.g., battery, photovoltaic cells, crank powered generator, power outlet, etc.), enabling the cold plasma treatment system 10 to be used in a wide variety of locations. In operation, the cold plasma treatment system 10 uses the controller 14 to produce an electrical signal. The electrical signal ionizes the atmospheric gases between a roller 34 and the patient treatment site 16 converting the atmospheric gases into a cold plasma. As the roller assembly 18 generates plasma the user (e.g., patient, doctor, physical therapist, trainer, patient, etc.) may repeatedly roll the roller assembly 18 back and forth over the patient treatment site 16 providing a massaging effect while simultaneously treating the treatment site 16 with cold plasma. Depending on the desired cold plasma treatment and/or massaging effect, the roller assembly 18 enables interchangeable attachment of different rollers 34. In some embodiments, the cold plasma applicator may 12 may include vibration elements 36 and/or heating elements 38 that aid in the treatment.

As illustrated, the controller 14 includes one or more processors 30 and one or more memories 32. In operation, the controller 14 uses the processor 30 to execute instructions stored in the memory 32 to produce and control the cold plasma generating electrical signal (e.g., change power, amplitude, frequency/frequencies, pulse timing, etc.), the vibration elements 36, and the heating elements 38. For example, the controller 14 may have preprogrammed modes that enable a user to select different modes of operation (e.g., a cold plasma and heat treatment; cold plasma and vibration treatment; cold plasma, heat, and vibration treatment; a treatment that cycles through cold plasma, heat, and vibration, etc.) for treating different injuries. In some embodiments, the electrical signal may be a multi-frequency harmonic-rich signal (e.g., a timed pulse electrical signal that is pulsed between 100-1000 Hz with an output voltage between 1-100 kV having multiple A/C waves at multiple frequencies that overlap to produce 2-2,000,000 or more harmonic components between DC and 500 MHz). As the multi-frequency, harmonic-rich electrical signal passes through the atmospheric gases; the gas molecules/atoms lose and gain electrons to produce cold plasma with positive ions, negative ions, and electrons. It is believed that the multi-frequency, harmonic-rich electrical signal facilitates removal of electrons from molecules/atoms with less energy than typical plasma formation. Accordingly, the plasma is a low temperature plasma or cold plasma (e.g., a cold plasma with a temperature between approximately 60-120, 60-80, 70-90, 80-100, 90-110, 100-120 degrees Fahrenheit), enabling exposure to a temperature sensitive target substrate (e.g., biological tissue).

FIG. 2 is a partial perspective view of an embodiment of the cold plasma applicator 12 without the roller 34. As illustrated, the roller assembly 18 may include a rib structure 50 that encloses a conductive rod 52 that couples to the wire 24. The rib structure 50 may include one or more structural supports or bars 54 (e.g., 1, 2, 3, 4, 5, or more) that extend between a first end cap 56 (e.g., annular end cap) and a second end cap 58 (e.g., annular end cap). In operation, the end caps 56 and 58 retain the bars 54 and rod 52 in a fixed arrangement, to provide structural support for the roller 34 while enabling interchangeability of the roller 34. In some embodiments, the end caps 56, 58 may include apertures 60 spaced about a circumference 62, wherein the apertures 60 receive the bars 54 and the conductive rod 52 to hold the bars 54 and the rod 52 in fixed positions relative to each other. The end cap 56 may also include a bearing (e.g., ball bearing, needle bearing, low friction material sleeve, etc.) or bearing surface 64 that enables the roller assembly 18 to rotate in circumferential directions 66, 68. In order to conduct the electrical signal from the conductive rod 52 to the roller 34, the roller assembly 18 may include an electrically conductive cotter pin 70 or another similar structure (e.g., electrical wiper) that electrically couples to the conductive rod 52 and one of the bars 54. In operation, the cotter pin 70 enables the electrical signal to transfer from the conductive rod 52 to the roller 34, enabling the roller 34 to produce cold plasma. In some embodiments, the roller assembly 18 may include one or more cotter pins 70 that couple to the same or different bars 54 enabling the electrical signal to reach the roller 34. In still other embodiments, the conductive rod 52 may be insulated except where the cotter pin 70 or similar structure couples to the conductive rod 52.

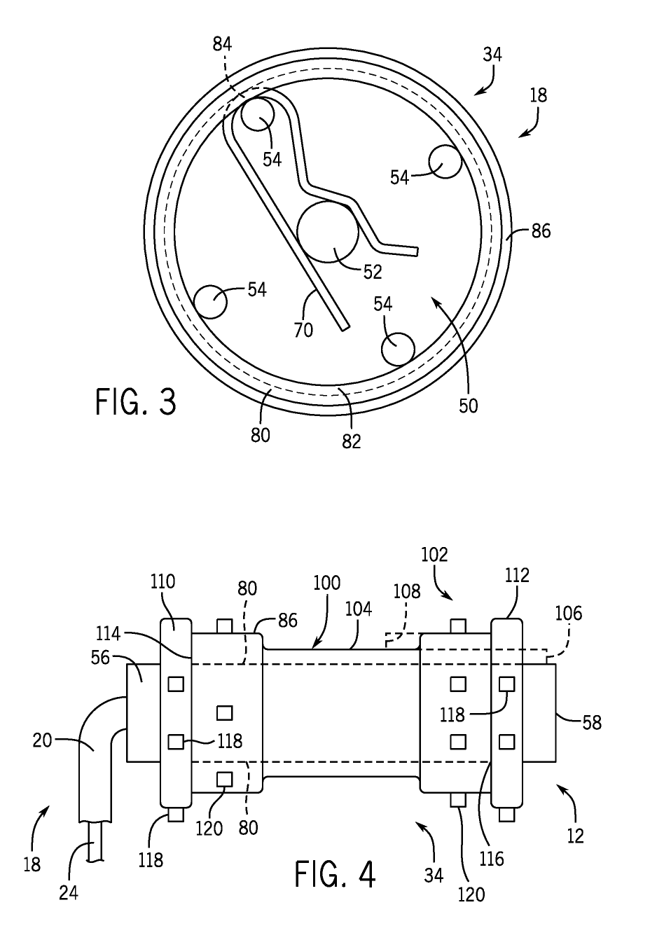

FIG. 3 is a cross-sectional view of an embodiment of the roller assembly 18 along line 3-3 of FIG. 2. As illustrated, the roller 34 includes a conductive layer 80 (e.g., annular metal layer or sleeve) that electrically couples to the conductive rod 52 via the cotter pin 70. For example, the conductive layer 80 may include a groove 82 (e.g., an annular metal groove) that receives an end 84 of the cotter pin 70, which maintains an electrical connection between the conductive layer 80 and the conductive rod 52 as the roller 34 rotates. Surrounding the conductive layer 80 is a dielectric layer 86 (e.g., annular dielectric layer or sleeve). The dielectric layer 86 may be a flexible dielectric that enables more effective treatment on a variety of patients and anatomical sites. For example, the dielectric layer 86 may be silicone, latex, open cell foam, hydrogels, polyoxymethylene, polyamide, polytetrafluoroethylene (PTFE), acetal homopolymer, polyethylene (PE), polypropylene (PP), poly vinyl chloride (PVC), ethylene vinyl acetate (EVA), propylene, copolyester ether, and polyolefin film.

In operation, the controller 14 provides an electrical signal that passes through the wire 24 (e.g., HV/RF feed cables) to the conductive rod 52. As the electrical signal passes through the conductive rod 52, the cotter pin 70 conducts the electrical signal to the conductive layer 80 where the electrical signal (e.g., multi-frequency, harmonic-rich electrical signal) builds charge. After enough charge builds on the conductive layer 80, the electrical signal passes through the dielectric layer 86 and the air between the patient (e.g., ground) and the roller 34. As the electrical signal crosses the air gap, the electrical signal forms cold plasma.

FIG. 4 is a side view of an embodiment of the cold plasma applicator 12. The roller 34 on the roller assembly 18 includes one or more cold plasma regions 100 and massage regions 102 (e.g., treatment regions 100 and 102). The cold plasma generating region 100 may be a recess 104 (e.g., annular recess) in the flexible dielectric layer 86 that enables air to flow between the roller 34 and the patient for cold plasma generation. As illustrated, the flexible dielectric barrier layer 86 has a thickness 106 next to the cold plasma generation region 100, while the rest of the dielectric barrier layer 86 has a thickness 108 in addition to the thickness 106. It is in this cold plasma generating region 100, where the dielectric barrier layer 86 has the thickness 106, that charge builds before crossing through the air gap. In other words, the dielectric barrier layer 86 has the additional thickness 108 to block charge movement except through the cold plasma generation region 100. As explained above, once a sufficient amount of charge builds on the dielectric barrier layer 86, the multi-frequency, harmonic-rich electrical signal crosses the air gap to the patient (e.g., ground), forming cold plasma. In some embodiments, the roller 34 may include electrically insulative end caps 110 and 112 that insulate ends 114 and 116 of the conductive layer 80. The insulative end caps 110 and 112 may also provide an additional massaging effect during use. For example, the insulative end caps 110 and 112 may include one or more mechanical massage features such as protrusions 118 (e.g., circumferential spaced protrusions) that facilitate or increase the massaging effect. In some embodiments, the dielectric barrier layer 86 may also include one or more mechanical massage features such as protrusions 120 (e.g., circumferential spaced protrusions) in the massaging regions 102 to facilitate or increase the massaging effect of the cold plasma applicator 12. In certain embodiments, the cold plasma applicator 12 may also vary the thickness/height and/or width of the electrically insulative end caps 110, 112 and/or dielectric barrier layer 86 to create various shapes that steady and center the cold plasma applicator 12 on various anatomical features (e.g., arm, leg, ankle, neck, etc.).

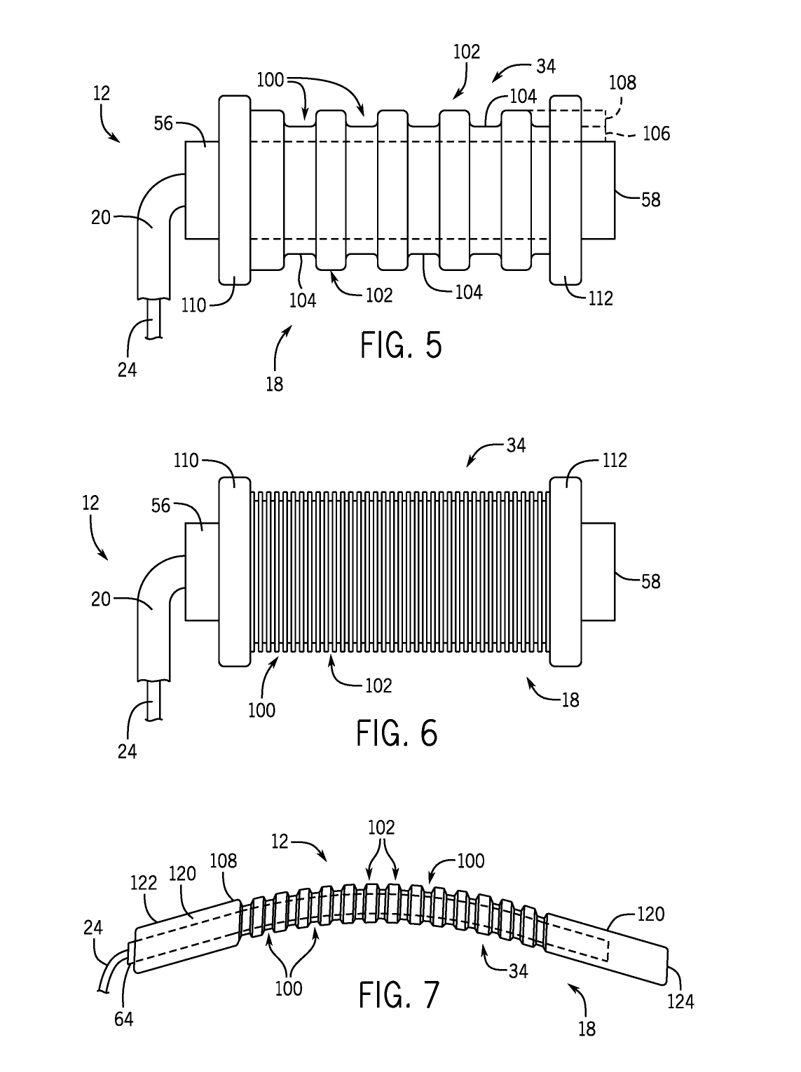

FIGS. 5 and 6 are side views of an embodiment of the cold plasma applicator 12. As illustrated, the roller 34 may include multiple cold plasma generating regions 100 between massaging regions 102 (e.g., in an alternating arrangement to distribute) cold plasma treatment and massage treatment across the length of the roller 34. In FIG. 5, the cold plasma generating regions 100 and massaging regions 102 are equally spaced apart to provide uniform massaging and cold plasma treatment. In some embodiments, the size, spacing, and/or number of the cold plasma generating regions 100 and the massaging regions 102 may be adjusted (e.g., increased or decreased) along the length of the roller 34. For example, the applicator 12 may be equipped with a smaller number, greater size, and greater spacing of the cold plasma generating regions 100 and massaging regions 102 across the roller 34 as depicted in FIG. 5, or the applicator 12 may be equipped with a greater number, smaller size, and smaller spacing of the cold plasma generating regions 100 and the massaging regions 102 as depicted in FIG. 6. In some embodiments, the cold plasma generating regions 100 and massaging regions 102 may be uniform or non-uniform in number, size, and/or spacing across the length of the roller 34. The cold plasma treatment system 10 may include multiple interchangeable rollers 34 for use with the applicator 12 enabling different types of massages and cold plasma treatments.

FIG. 7 is a side view of an embodiment of a cold plasma applicator 12. In contrast, to the cold plasma applicators 12 in the previous figures, the cold plasma applicator 12 in FIG. 7 includes a roller 34 with two handles 120 formed out of a flexible dielectric material (e.g., silicone, latex, open cell foam, hydrogels, polyoxymethylene, polyimide, polytetrafluoroethylene (PTFE), acetal homopolymer, polyethylene (PE), polypropylene (PP), poly vinyl chloride (PVC), ethylene vinyl acetate (EVA), propylene, copolyester ether, and polyolefin film) to form a flexible cold plasma applicator 12 that conforms to a treatment site. However, in some embodiments the dielectric material may be stiff forming a rigid cold plasma applicator 12. As illustrated, the two handles 120 are on opposite ends 122 and 124 of the roller 34 with a conductive wire 24 passing between the two handles 120. In some embodiments, the cold plasma applicator 12 may include a bearing 64 that enables the roller 34 to rotate with respect to the wire 24. In operation, a user may grab both ends 122, 124 of the cold plasma applicator 12 to apply pressure during a massaging treatment, while the conductive wire 24 delivers the electrical signal to the multiple cold plasma generating regions 100. As explained above, once a sufficient amount of charge builds on the dielectric material in the cold plasma generating regions 100, the multi-frequency, harmonic-rich electrical signal crosses the air gap to the patient (e.g., ground), forming cold plasma. Separating the cold plasma generating regions 100 are massaging regions 102. As explained above, the number, size, and/or spacing of the cold plasma generating regions 100 and massaging regions 102 may be adjusted (e.g., increased or decreased) along the length of the roller 34. In some embodiments, the cold plasma generating regions 100 and massaging regions 102 may be uniform or non-uniform in size, spacing, and/or number across the length of the roller 34. These variations enable different types of cold plasma and massage treatments with different cold plasma applicators 12.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A system comprising:
a cold plasma treatment system, comprising:
a controller configured to produce an electrical signal that generates cold plasma;
a cold plasma applicator coupled to the controller, comprising:
a roller with at least one cold plasma generating region and at least one massage region, wherein the cold plasma applicator is configured to provide a cold plasma treatment and a massage treatment with the at least one cold plasma generating region and a massage treatment with the at least one massage region.

2. The system of claim 1, wherein the cold plasma applicator comprises a rib structure, structurally supporting the roller.

3. The system of claim 2, wherein the rib structure surrounds a conductive rod configured to electrically couple to the roller.

4. The system of claim 3, wherein the conductive rod is configured to electrically couple to the roller.

5. The system of claim 3, wherein the cold plasma applicator comprises a first handle coupled to the rib structure.

6. The system of claim 5, wherein the handle has an internal passage with a wire extending through and coupling to the conductive rod.

7. The system of claim 1, wherein the roller comprises a conductive layer surrounded by a dielectric layer.

8. The system of claim 7, wherein a recess in the dielectric layer forms the at least one cold plasma generating region.

9. The system of claim 1, wherein the at least one massage region comprises at least one protrusion.

10. The system of claim 1, wherein the roller comprises a first insulative end cover and a second insulative end cover on respective first and second ends of the roller.

11. The system of claim 1, comprising a first handle and a second handle on respective first and second ends of the roller.

12. The system of claim 1, comprising a portable housing that contains the controller and a power source, wherein the portable housing couples to the cold plasma applicator.

13. A system comprising:
a cold plasma treatment system, comprising:
a portable housing, comprising:
a controller configured to produce an electrical signal that generates cold plasma; and
a power source coupled to the controller;
a cold plasma applicator coupled to the housing, wherein the cold plasma applicator comprises:
a roller with at least one cold plasma generating region and at least one massage region, wherein the cold plasma applicator is configured to provide a cold plasma treatment with the at least one cold plasma generating region and a massage treatment with the at least one massage region.

14. The system of claim 13, wherein the roller comprises a conductive layer surrounded by a dielectric layer.

15. The system of claim 14, wherein a recess in the dielectric layer forms the at least one cold plasma generating region.

16. The system of claim 13, wherein the cold plasma applicator comprises a rib structure, structurally supporting the roller.

17. The system of claim 16, wherein the rib structure surrounds a conductive rod configured to electrically couple to the roller.

18. A method comprising:
controlling generation of a cold plasma with a cold plasma applicator, wherein the cold plasma applicator includes a cold plasma region and a massage region.

19. The method of claim 18, comprising distributing the cold plasma into a plurality of cold plasma regions disposed adjacent a plurality of massage regions. The method of claim 18, wherein the massage region comprises one or more protrusions that facilitate the massage treatment.

21. The method of claim 18, comprising rotating a roller of the cold plasma applicator, wherein the roller includes the cold plasma region and the massage region.
